(19) United States
(12) Patent Application Publication
(10) Pub. No.: US 2009/0093440 A1
(43) Pub. Date: Apr. 9, 2009

(54) Fragranced Therapeutic Delivery System

(76) Inventor: Howard Murad, Marina Del Rey, CA (US)

Correspondence Address: LOUIS C. PAUL, 420 East 61st Street, 8E, NEW YORK, NY, 10021, US

(21) Appl. No.: 12/086,911

(22) PCT Filed: Dec. 20, 2006
(86) PCT No.: PCT/US06/48383

§ 371 Date: Jun. 20, 2008

Related U.S. Application Data
(60) Provisional application No. 60/751,635, filed on Dec. 20, 2005.

(51) Int. Cl. A61K 31/715; A61K 31/20; A61K 31/661; A61K 31/4015; A61P 17/00

(52) U.S. Cl. 514/55; 514/558; 514/143; 514/423

(57) ABSTRACT

The present invention relates to multi-functional topical delivery systems for providing long-lasting delivery of fragrance as. well as skin-supporting and/or pharmaceutically active ingredients comprising (i) an oil phase; (ii) an aqueous phase; (iii) phenoxyethanol at a concentration of from about 2.0% to about 2.7% based on the total weight of the composition; (iv) an effective exfoliating amount of a hydrophobic hydroxycarboxylic acid selected from the group consisting of orthohydroxybenzoic acid, hydroxycarboxylic acids containing a C12-C24 fatty acid esterified to the alpha carbon hydroxyl group, hydroxycarboxylic acids containing a C12-C24 fatty alcohol esterified to a carboxyl group; (v) a non-ionic emulsifier having an HLB of from about 7 to about 10; (vi) a fragrance composition; and (vii) at least one skin-supporting or dermatopharmaceutically active agent.

### FIELD OF INVENTION

The present invention relates to multi-functional topical delivery systems for providing long-lasting delivery of fragrance as well as skin-supporting and/or pharmaceutically active ingredients.

### BACKGROUND

Fragrance compositions are blends of perfumery ingredients (essential oils, concretes and extracts and other aroma-producing chemicals, both natural and synthetic) that, in specified proportions, produce distinct, recognizable, desired scents. They have long been incorporated into topical compositions, both prescription and over-the-counter dermatologic products as well as in cosmetics not only for the purpose of masking odors characteristic of certain active ingredients but also for their own therapeutic benefits (e.g., aromatherapy). From the standpoint of users of topical skin treatment products, a need has existed, and continues to exist, for fragranced skin care products that, upon application, provide a continuous, uniform release of fragrance, imparting the desired scent over an extended period of time while at the same time effectively delivering skin-supporting or pharmaceutically active ingredients.

The difficulty in achieving these objectives lies in the volatile nature of fragrance compositions. In order for fragrances to be effective, they must be volatilized—small and light enough to enter the nasal passage, be trapped by cilia and trigger one of hundreds of different olfactory receptors. This very volatility, however, makes fragrance short-lived.

Upon application to the skin (or hair), fragrance compositions not only lose their intensity but also change in character. This is due, in part, to differential evaporation and surface penetration of the various perfumery ingredients which, in combination, produce the characteristic notes of a fragrance (e.g., top, middle and bottom). Thus, it is well-known in the art, that in order to maintain a desired fragrance the more fleeting top notes (such as those produced by ingredients derived from citrus plants) must be released more frequently than longer-lasting middle and bottom notes.

One approach to extending the longevity of fragrance has been to slow the release of individual perfumery ingredients by “holding” the fragrance as a whole on the skin for a longer period of time. Another approach has been to blend the fragrance composition with a polymeric substance that will entrap the perfumery materials so that small amounts are released over time. A further approach is to deliver the perfumery ingredients in an essentially scent-free form that upon contacting the acid mantle of the skin releases the fragrance. The latter approach is illustrated by U.S. Pat. Nos. 5,378,468 and 6,576,247. All documents cited are, in relevant, part, incorporated herein by reference.

The use of penetration enhancers to increase the efficacy of topically-applied compositions in delivering active ingredients is well-known in the art. See, e.g., E W Smith and H I Maibach (eds.), Percutaneous Penetration Enhancers, 2^{nd} edition (Boca Raton, Fla.: Taylor & Francis 2005).

The use of hydroxy-acids in the treatment of photodamaged skin and other skin conditions is well-known in the cosmetic and dermatologic arts. See, H. Murad, The Murad Method, pp. 71-76 (2003). See also, C M Dietre, “Effects of alpha-hydroxy acids on photoaged skin,” J. Am. Acad. Dermatol. Vol. 34, pp. 187-195 (1996); E. Berardesca, “AHA mechanism of action,” Cosmet. & Toiletries, Vol. 110, pp. 30-31 (1995). Hydroxy acids used in skin care products are, generally classified into categories, based on similarities in their chemical structure: alpha hydroxy, beta hydroxy and poly hydroxy.

Alpha hydroxy acids (AHAs) are linear, aliphatic, and water-soluble. This group is subdivided into three sub-classes: monocarboxylic (glycolic, lactic, mandelic); dicarboxylic (malic and tartaric); and tricarboxylic (citric). The most immediate effect of AHAs is corneocyte disadhesion, specifically in the stratum corneum. Longer onset effects reported to be associated with AHAs include increased synthesis of glycosaminoglycans. However, dermal irritation, clinically manifested as stinging and burning, is a well-known side effect associated with penetration of AHAs into the dermis. U.S. Patent Publication 2003/0027833, Paragraphs 0065-0066 teaches the use of citric acid as a penetration enhancer at “an effective enhancing amount”, which is defined as from about 0.1% to about 20%, more preferably from about 1% to about 10%.

Salicylic acid is a beta hydroxy acid (BHA). It is a phenolic, hydrophobic compound, that induces exfoliation, including in sebaceous areas. Salicylic acid is also a comedolytic approved by the FDA for the treatment of acne. Due to its lipophilicity, salicylic acid has a lower degree of dermal penetration than AHAs such as glycolic acid. U.S. Patent Publication No. 2004/0076648, Paragraphs 133-136 teaches the use of salicylic acid as a percutaneous penetration enhancer at concentrations preferably from about 1% to about 10% by weight of the total composition weight, more preferably from about 2% to about 5% by weight. See also, U.S. Patent Publication 2003/0027833, Paragraphs 0065-0066 (teaching salicylic acid at levels of from about 0.1% to about 20%, more preferably from about 1% to about 10%).

Polyhydroxy acids (PHAs) are larger molecular weight compounds in comparison to AHAs. They are known in the art to penetrate less rapidly and less deeply into the dermis, thus resulting in less dermal irritation than AHAs.

It well-known in the art that the acid mantle—the acidic, hydrolipid film on the skin outermost layers—provides a protective barrier, helping to maintain the skin's strength and integrity and to ward off infections by preventing the growth of bacteria and fungi. The physiological pH the acid mantle in normal healthy skin has an average value of between 4 and 6. See, e.g., Rippke F, et al., “The acidic milieu of the horny layer: new findings on the physiology and pathophysiology of skin pH,” Am. J. Clin. Dermatol. 3(4):261-72 (2002). It is also well-known among those skilled in the art that the efficacy of topically-applied compositions, particularly those containing hydroxy acids, can be dependent on the pH of the acid mantle.

The prior art teaches compositions purporting to provide improved fragrance longevity that also contain active ingredients. For example, U.S. Pat. No. 6,893,647 discloses cosmetic compositions where fragrance and cyclic oligosaccharides having one or more unsubstituted alkyl substituents complex to provide longer lasting fragrance. These compositions are also taught to include a wide variety of additional ingredients for providing both aesthetic and usage benefits. Among these additional ingredients are alcohol denaturants, UV stabilizers, antioxidants, preservatives, dyes, pH adjusting agents, antimicrobials, deodorants, humectants and other skin conditioning agents. The preservatives disclosed in the '647 Patent are commonly-used in topical formulations and include phenoxyethanol, benzyl alcohol, methyl paraben and propyl paraben. As suitable pH adjusting agents, two AHAs (lactic acid and citric acid) are listed along with sodium citrate, succinic acid, phosphoric acid, sodium hydroxide and sodium carbonate.

The use of essential oils and other fragrance ingredients in topically-applied therapeutic compositions is well-known in the art. For example, U.S. Pat. No. 5,599,546 teaches medicinal masks for treating a variety of dermatologic conditions (dry skin, dermatosis, acne, keratosis, photo-aging, melasma, itching, inflammation and folliculitis barbae). The masks, which are described as degreasing and treating the skin, while at the same time providing for time release of a therapeutic agent, consist essentially of three components: (a) from greater than zero to up to about 20% by volume of alpha hydroxy acids, carboxylic acids, halo-carboxylic acids, dicarboxylic acids, and combinations thereof; (b) from 0.1% to 2% by volume of a limonene-based oil and (iii) from greater than zero to 40% by volume of a mineral-based skin-absorbent carrier (i.e., kaolin, magnesium aluminum silicate, bentonite and combinations thereof).

In a preferred embodiment, the '546 Patent discloses the use of 5% to 10% by volume of glycolic acid as a preferred AHA and 0.1% to 0.2% of lemon oil as a preferred limone-based oil. In addition to lemon oil, the '546 Patent discloses orange oil. grapefruit oil, lime oil and bergamot oil as suitable limonene-based oils. The '546 Patent also teaches that perfumes, preservatives and non-aqueous solvents may be incorporated in the claimed compositions. Isopropyl and SDA-40 alcohols are disclosed as suitable non-aqueous solvents. Methyl ethyl paraben and propylparaben are taught as preservatives that may be included. Phenoxyethanol is not, however, disclosed. The weight percentages of the non-aqueous solvent and preservatives are not disclosed.

Phenoxyethanol is an aromatic ether alcohol. In the cosmetic and personal care arts, it is mostly commonly used as a preservative. See, Cosmetic, Toiletries & Fragrance Association, International Cosmetic Ingredient Dictionary and Handbook, Vol. II, p. 1364 (10^{th} Edition, 2004) (“CTFA Dictionary”). Phenoxyethanol is also known to those of skill in the art as a fragrance ingredient and as a penetration enhancer. U.S. Pat. No. 5,374,661, for example, teaches the use of ether alcohols and fatty alcohol esters to enhance the transdermal: permeation of diclofenac, a non-steroidal anti-inflammatory drug. Preferred ether alcohols taught in the '661 Patent include butoxydiglycol, ethoxyethanol, methoxyethanol, phenoxydiglycol, phenoxyethanol, phenoxyisopropanol, methoxypropanol and methoxydiglycol, the most preferred being ethoxyldiglycol.

Applicants have surprisingly discovered that an oil-in-water emulsion comprising phenoxyethanol at a concentration of from about 2.0% to about 2.7% in combination with an effective exfoliating amount of a hydrophobic hydroxycarboxylic acid, most preferably orthohydroxybenzoic acid, provides both long-lasting fragrance release as well as a highly efficacious vehicle for topical deliver of skin-supporting and/or dermatopharmaceutically active agents.

### SUMMARY OF THE INVENTION

The present invention relates to a multi-functional system for topical delivery of a fragrance composition and one or more active ingredients in a dermatologically acceptable carrier. More particularly, the invention relates to an oil-in-water emulsion topical delivery system comprising (i) an oil phase; (ii) an aqueous phase; (iii) phenoxyethanol at a concentration of from about 2.0% to about 2.7% based on the total weight of the composition; (iv) an effective exfoliating amount of a hydrophobic hydroxycarboxylic acid selected from, the group consisting of orthohydroxybenzoic acid, hydroxycarboxylic acids containing a C₁₂-C₂₄ fatty acid esterified to the alpha carbon hydroxyl group, hydroxycarboxylic acids containing a C₁₂-C₂₄ fatty alcohol esterified to a carboxyl group; (v) a non-ionic emulsifier having an HLB of from about 7 to about 10; (vi) a fragrance composition; and (vii) at least one skin-supporting or dermatopharmaceutically active agent.

### DETAILED DESCRIPTION OF THE INVENTION

The present invention relates an oil-in-water emulsion topical delivery system comprising (i) an oil phase; (ii) an aqueous phase; (iii) phenoxyethanol at a concentration of from about 2.0% to about 2.7% based on the total weight of the composition; (iv) an effective exfoliating amount of a hydrophobic hydroxycarboxylic acid selected from the group consisting of orthohydroxybenzoic acid, hydroxycarboxylic acids containing a C₁₂-C₂₄ fatty acid esterified to the alpha carbon hydroxyl group, hydroxycarboxylic acids containing a C₁₂-C₂₄ fatty alcohol esterified to a carboxyl group; (v) a non-ionic emulsifier having an HLB of from about 7 to about 10; (vi) a fragrance composition; and (vii) at least one skin-supporting or dermatopharmaceutically active agent.

Phenoxyethanol

Phenoxyethanol is an aromatic ether alcohol having the empirical formula C₈H₁₀O₂. Other technical names of phenoxyethanol include ethylene glycol monophenyl ether and 2-hydroxyethyl phenyl ether. It is an article of commerce well-known to those of skill in the art and available from a number of commercial sources including those listed in the CTFA Dictionary, Vol. II, pp. 1364-1365.

In the delivery system of the present invention, phenoxyethanol is present at concentrations ranging from about 0.1% to about 5%, preferably from about 0.2% to about 3%, and more preferably from about 0.3% to about 2.5%.

Hydrophobic Hydroxycarboxylic Acid

Hydrophobic hydroxycarboxylic acids suitable for use in the topical delivery system of the present invention are selected from the group consisting of orthohydroxybenzoic acid, hydroxycarboxylic acids containing a C₁₂-C₂₄ fatty acid esterified to the alpha carbon hydroxyl group, hydroxycarboxylic acids containing a C₁₂-C₂₄ fatty alcohol esterified to a carboxyl group. In a preferred embodiment, the hydrophobic hydroxycarboxylic acid is present at a concentration of at least about 0.5%. In a particularly preferred embodiment, the hydrophobic hydroxycarboxylic acid is orthohydroxybenzoic acid.

Hydrophilic Hydroxyacids Acids

In another aspect of the present invention, the topical delivery system comprises both a hydrophobic hydroxycarboxylic acid and a hydrophilic hydroxycarboxylic acid. Hydrophilic hydroxycarboxylic acids suitable for use in the present invention include alpha hydroxy acids (AHAs) and polyhydroxyacids (PHAs).

AHAs are a group of hydroxy acids in which the hydroxy group is attached to the alpha carbon atom of the acid. They conform to the structure: (R₁) (R₂) C (OH) COOH, where R₁ and R₂ are selected from the group consisting of hydrogen, alkyl, aralkyl and aryl groups, the latter groups having 1-29 carbon atoms. The alkyl, aralkyl and aryl groups may be saturated or unsaturated, isomeric or non-isomeric, straight or branched chain or cyclic. The alkyl, aralkyl and aryl groups may also contain as substituents OH, CHO, COOH and alkoxy groups having 1 to 9 carbon atoms. In addition, R₁ and R₂ may also Cl, Br, I, S, F, or an alkyl or alkoxy group, saturated or unsaturated, having 1 to 9 carbon atoms.

As used in the present application, the term “AHA” means the free acid, its corresponding ester (formed by reaction of the AHA with an alcohol), its corresponding lactone (formed by the reaction of the carboxylic acid and hydroxyl groups of the AHA), as well as its corresponding salt (formed by reaction of the AHA with an organic base or an inorganic alkali). R₁ and R₂ may be the same or different. In the latter case, the AHAs may be stereoisomers in the D, L, and DL forms. AHAs suitable for use in the present invention may be grouped into (i) alkyl AHAs, (ii) aralkyl and aryl AHAs, (iii) polyhydroxy AHAs, and (iv) polycarboxylic AHAs.

Alkyl AHAs (i.e., where R₁ and R₂ are hydrogen or alkyl) suitable for use in the present invention include: 2-hydroxyethanoic acid (glycolic acid, hydroxyacetic acid); 2-hydroxypropanoic acid (lactic acid): 2-methyl 2-hydroxypropanoic acid (methyllactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxynonanoic acid; 2-hydroxydecanoic acid; 2-hydroxyundecanoic acid; 2-hydroxydodecanoic acid (alpha hydroxylauric acid); 2-hydroxytetradecanoic acid (alpha hydroxymyristic acid); 2-hydroxyhexadecanoic acid (alpha hydroxypalmitic acid); 2-hydroxyoctadecanoic acid (alpha hydroxystearic acid); 2-hydroxyeicosanoic acid (alpha hydroxyarachidonic acid).

Aralkyl and aryl AHAs (i.e., where R₁ and R₂ are arylalkyl or aryl) suitable for use in the present invention include: 2-phenyl 2-hydroxyethanoic acid (mandelic acid); 2,2-diphenyl 2-hydroxyethanoic acid (benzilic acid); 3-phenyl 2-hydroxypropanoic acid (phenyllactic acid); 2-phenyl 2-methyl 2-hydroxyethanoic acid (atrolactic acid, 2-(4′-hydroxyphenyl); 2-hydroxyethanoic acid (4-hydroxymandelic acid); 2-(4′-chlorophenyl) 2-hydroxyethanoic acid (4-chloromandelic acid); 2-(3′-hydroxy4′-methoxyphenyl) 2-hydroxyethanoic acid (3-hydroxy-4-methoxymandelic acid); 2-(4′-hydroxy-3′-methoxyphenyl); 2-hydroxyethanoic acid (4-hydroxy-3-methoxymandelic acid); 3-(2′-hydroxyphenyl); 2-hydroxypropanoic acid (3-(2′-hydroxyphenyl) lactic acid); 3-4′-hydroxyphenyl) 2-hydroxypropanoic acid (3-(4′-hydroxyphenyl) lactic acid)); 2-(3′,4′-dihydroxyphenyl) 2-hydroxyethanoic acid (3,4-dihydroxymandelic acid).

Polyhydroxy AHAs suitable for use in the present invention include: 2,3-dihydroxypropanoic acid (glyceric acid); 2,3,4-trihydroxybutanoic acid and its isomers (erythronic acid, threonic acid); 2,3,4-tetrahydroxypentanoic acid and its isomers (ribonic acid, arabinoic acid, xylonic acid, lyxonic acid); 2,3,4,5,6-pentahydroxyhexanoic acid and its isomers (allonic acid, altronic acid, gluconic acid, mannoic acid, gulonic acid, idonic acid, galactonic acid, talonic acid); 2,3,4,5,6,7-hexahydroxyheptanoic acid and its isomers (glucoheptonic acid, galactoheptonic acid)

Polycarboxylic AHAs suitable for use in the present invention include: 2-hydroxypropane-1,3-dioic acid (tartronic acid); 2-hydroxybutane-1,4-dioic acid (malic acid); 2,3-dihydroxybutane-1,4-dioic acid (tartaric acid); 2-hydroxy-2-carboxypentane-1,5-dioic acid (citric acid); 2,3,4,5-tetrahydroxyhexane-1,6-dioic acid and its isomers (saccharic acid, mucic acid).

In a preferred embodiment of the delivery system of the present invention, the AHA is monocarboxylic and is selected from the group consisting of glycolic acid, lactic acid, and mandelic acid.

Glycolic acid conforms to the formula HOCH₂COOH. It is an article of commerce well-known to those of skill in the art and is available from a number of commercial sources including those listed in the CTFA Dictionary, Vol. I, pg. 755.

Lactic acid conforms to the formula:

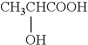

It is an article of commerce well-known to those of skill in the art and available from a number of commercial sources including those listed in the CTFA Dictionary, Vol. II, pg. 942.

Mandelic acid conforms to the empirical formula C₈H₈O₃. It is an article of commerce well-known to those of skill in the art and is available from a number of commercial sources including those listed in the CTFA Dictionary, Vol. II, pg. 1025.

In another preferred embodiment of the delivery system of the present invention, the AHA is polycarboxylic and is selected from the group consisting of malic acid, tartaric acid and citric acid.

Malic acid conforms to the structure:

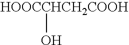

It is an article of commerce well-known to those of skill in the art and is available from a number of commercial sources including those listed in the CTFA Dictionary, Vol. II, pp. 1019-1020.

Tartaric acid conforms to the structure:

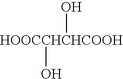

It is an article of commerce well-known to those of skill in the art and is available from a number of commercial sources including those listed in the CTFA Dictionary, Vol. II, pp. 1019-1020.

Citric acid conforms to the following structure:

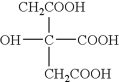

It is an article of commerce well-known to those of skill in the art and available from a number of commercial sources including those listed in the CTFA Dictionary, Vol. I, pp. 412-413.

In another embodiment of the delivery system of the present invention, the hydroxy acid is a polyhydroxy acid. In a preferred embodiment, the polyhydroxy acid is selected from the group consisting of gluconolactone and lactobionic acid.

Hydrophilic hydroxycarboxylicacids are used in the delivery systems of the present invention at concentrations ranging from about 0.1% to about 6%, preferably from about 0.2% to about 4%, and more preferably from about 0.5% to about 3%.

Fragrance Composition

The formation of a distinct, ambient aroma—one possessing a desired, recognizable character—is achieved by blending perfumery materials of defined quality In specified proportions. As used in the present application, the term “fragrance composition” refers to a blend of individual perfumery materials, each with a characteristic odor or “note”. More particularly, fragrance compositions are made up of “accords”—mixtures of two or more perfumery materials having a unified olfactory theme.

A widely used methodology for constructing fragrance compositions, well-known to those of skill in the art, is to combine accords starting with those of the lowest volatility (“base notes”), then adding ingredients of intermediate volatility or tenacity (“middle notes”) and lastly adding the most volatile materials (“top notes”).

Top notes are scents that are perceived within the first few minutes of release of the fragrance composition from the skin or hair. They are lighter scents, usually lasting from about five to ten minutes. Representative top notes include those classified as “citrus” such as grapefruit, lemon, orange or bergamot, or those classified as “herbaceous” such as mint, rosemary, or sage.

Middle notes are the scents that emerge after the top notes evaporate. These notes form the “heart” or main body of a scent; they smooth or round out the initial sharpness caused by the top notes and last for up to one hour. Typically, middle notes emerge fifteen minutes after release of the top note(s). Representative of middle notes are lavender, juniper or chamomile.

Base notes are usually not perceived until about one hour after the release of the fragrance composition, and may last for several hours. Representative base notes include woody or musk notes such as sandalwood, synthetic musk (civet) or vetiver.

Perfumery materials are well-known in the art and include essential oils, concretes and extracts and aroma-producing chemicals, which may be of natural or synthetic origin. Chemically, perfumery materials may be aliphatic and aromatic alcohols, aldehydes, ketones, aliphatic and aromatic esters, nitrites, ethers, lactones, heterocyclics or terpenes. They are obtained by techniques that are well-known to those of skill in the art—essential oils by steam distillation or crushing; concretes by extraction with volatile solvents; absolutes by extraction of concretes with alcohol.

Natural, botanical sources of perfumery ingredients useful in the present invention are often grouped into the following families: flowers and blossoms; leaves and twigs; roots, rhizomes and bulbs; seeds; fruits and fruit peels; woods; barks; resins and saps; lichens and mosses.

The fragrance family of flowers and blossoms are understood by those of skill in the art to include bergamot; clove; citrus including orange (neroli), lemon, lime, grapefruit, mandarin; geranium; jasmine; lavender; orchid; osmanthus or mimosa genera; plum, rose; tuberose; Ylang-ylang.

The fragrance family of leaves and twigs are understood to include citrus leaves; patchouli; rosemary; sage; thyme.

The fragrance family of roots, rhizomes and bulbs includes the rhizomes from iris and ginger, and roots of vetiver.

The fragrance family of seeds includes anise, caraway, cardamom, cocoa, coriander, cumin, mace, nutmeg, pepper, tonka bean, vanilla.

The fruit fragrance family includes apple, banana, cherry, melon, peach, pineapple, strawberry, citrus rinds including orange, lemon, lime, grapefruit, mandarin.

The woods fragrance family includes agarwood, birch, cedar, juniper, pine, rosewood, sandalwood.

The bark fragrance family includes cascarilla, cinnamon, sassafras.

The fragrance family of resins and saps includes amber, copal, fir, frankincense, gum benzoin, labdanum, myrrh, peru balsam, pine.

The fragrance family of lichens and mosses includes oakmoss and treemoss.

In the past, certain families of fragrance materials were obtained from animals. The family of musks was derived from musk deer, muskrat, musk ox. The family of civet was obtained from various species of the family Viverridae, including civets, genets and linsangs. Castoreum was obtained from the North American beaver. Ambergris was obtained from the sperm whale. Today, synthetic perfumery ingredients are created through organic chemical processes to mimic these animal-derived materials.

Synthetic perfumery ingredients are also created to mimic fragrances derived from botantical sources, as well as to create fragrances not typically found in natural sources.

Perfumery ingredients from the above-described fragrance families are combined into accords, which in turn, are used to build the top, middle and bottom notes of a fragrance. A typical fragrance Composition is comprised of about 25% top notes, about 25% middle notes and about 50% bottom notes. The individual perfumery materials and accords suitable for use in the delivery system of the present invention are catalogued and described in references and databases well-known to those of skill in the art including the following: S. Arctander, Perfume and Flavor Chemicals, Volumes I & II (1960, 1969; reprinted 2000); Allured's Flavor and Fragrance Materials (2005); the database maintained by the Research Institute for Fragrance Materials at www.rifm.org.

In delivery systems of the present invention a fragrance composition is present in an amount such that the desires fragrance is organoleptically perceptible for an extended period of time after application of the delivery system to the skin or hair. Where the fragrance composition is present as an undiluted essential oil, it is typically present at a concentration of at least about 0.1%. In a preferred embodiment, the fragrance composition undiluted and is present at a concentration of at least about 0.5%.

Skin-Supporting and Dermatopharmaceutically Active Ingredient

In one embodiment, the delivery system of the present invention includes a skin-supporting ingredient. As used in the present application, “skin-supporting ingredient” means one of a group of ingredients that help prevents skin cells from losing water, more particularly by increasing intracellular water content. Non-limiting examples of skin-supporting ingredients include: ceramides; glycosaminoglycans, as well as their primary component, n-acetyl glucosamine; botanical oils rich in C₁₆-C₂₀ fatty acids; phospholipids; amino acids; glycerols; phospholipids; glycosphingolipids; sodium PCA (pyrrolidone carboxylic acid).

Preferred glycosaminoglycans are hyaluronic acid and chondroitin sulfate.

Preferred phosholipids are lecithin and/or its components choline and phosphatidylcholine.

In one preferred embodiment, the botantical oil is rich in C₁₈ fatty acid(s), particularly those C₁₈ fatty acid(s) having at least two carbon-carbon double bonds.

In one preferred embodiment, the C₁₈ fatty acid has three carbon-carbon double bonds, each in the cis orientation. Alpha-linolenic acid (all-cis-9,12,15-octadecatrienoic acid) is also known as an omega-3 fatty acid. Flax seed oil, canola oil and soybean oil are preferred skin-supporting ingredients that are rich in omega-3 fatty acid. Gamma-linolenic acid (all-cis 6,9,12-octadecatrienoic acid) is also known as an omega-6 fatty acid. Black currant oil, evening primrose oil, and borage oil are preferred skin-supporting ingredients that are rich in omega-6 fatty acid. Linoleic acid (cis-cis-9,12-octadecadienoic acid) is also an omega-6 fatty acid. Grape seed oil is a preferred skin-supporting ingredient that is rich in omega-6 fatty acid.

In another preferred embodiment, the C₁₈ fatty acid has one carbon-carbon double bond. Oleic acid (9-octadecenoic acid) is known as an omega-9 fatty acid. Olive oil is a particularly preferred skin-supporting active ingredient that is rich in omega-9 fatty acid.

The CTFA Dictionary describes a wide variety of non-limiting cosmetic and pharmaceutical ingredients commonly used in the skin care industry, which are suitable for use in the delivery system of the present invention. Examples of these ingredient classes include: abrasives, absorbents, astringents, anti-acne agents, antimicrobial agents, antioxidants, external analgesics, film formers or materials (e.g., polymers, for aiding the film-forming properties and substantivity of the composition), humectants, moisturizers, pH adjusters, skin bleaching and lightening agents, skin-conditioning agents, skin soothing and/or healing agents, vitamins and derivatives thereof. Other examples of cosmetic and/or pharmaceutical ingredients which are suitable for use in the delivery system of the present invention are disclosed in U.S. Pat. No. 6,492,326.

Non-limiting examples of anti-acne ingredients which may be topically delivered in the present invention include: resorcinol, sulfur, salicylic acid, benzoyl peroxide, erythromycin, and zinc. Further examples of suitable anti-acne actives are described in U.S. Pat. No. 5,607,980.

Non-limiting examples of skin bleaching and lightening agents which may be topically delivered in the present invention include: arbutin, hydroquinone, kojic acid, ascorbic acid, magnesium ascorbyl phosphate and ascorbyl glucosamine.

Non-limiting examples of antioxidants/radical scavengers which may be topically delivered in the present invention include: ascorbic acid (vitamin C) and its salts; ascorbyl esters of fatty acids, ascorbic acid derivatives (e.g., magnesium ascorbyl phosphate, sodium ascorbyl phosphate, ascorbyl sorbate); tocopherol (vitamin E), tocopherol sorbate, tocopherol acetate, other esters of tocopherol; butylated hydroxybenzoic acids and their salts; 6-hydroxy-2,5,7,8-tetramethylchroman-2-carboxylic acid; gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione); coenzyme Q10 and its analogues, including without limitation, idebenone; dihydroxyfumaric acid and its salts; lycine pidolate, arginine pilolate; nordihydroguaiaretic acid; bioflavonoids; curcumin, lysine; 1-methionine; proline; superoxide dismutase; silymarin; tea extracts; grape skin/seed extracts; melanin; and rosemary extracts.

Non-limiting examples of steroidal anti-inflammatory agents which may be topically delivered in the present invention include: hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Non-limiting examples of non-steroidal anti-inflammatory agents which may be topically delivered in the present invention include: (i) oxicams, such as piroxicam, isoxicam, tenoxicam, and sudoxicam; (ii) salicylates, such as aspirin, disalcid, benorylate, trilisate, safapryn, solprin, diflunisal, and fendosal; (iii) acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; (iv) fenamates, such as mefenamic, meclofenamic, flufenamic, niflumic, and tolfenamic acids; (v) propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and (vi) pyrazoles, such as phenylbutazone, oxyphenbutazone, feprazone, azapropazone, and trimethazone.

Non-limiting examples of antimicrobial and antifungal agents suitable for use in the present invention include: β-lactam agents, quinolone agents, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxylsopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Non-limiting examples of anti-cellulite agents suitable for use in the present invention include xanthine compounds such as caffeine, theophylline, theobromine, and aminophylline.

Non-limiting examples of skin soothing and/or healing agents suitable for use in the present invention include: panthenol and derivatives, aloe vera and its derivatives, pantothenic acid and its derivatives, allantoin, bisabolol, and dipotassium glycyrrhizinate.

Other pharmaceutically-active ingredients that are known to be capable of transdermal delivery may be used the delivery system of the present invention.

In one embodiment, the pharmaceutically-active ingredient is a steroidal reproductive agent, non-limiting examples of which include: androgens, such as, for example, androstenediol and androisoxazole (for anabolic disorders), testosterone (hypogonadism, muscle wasting, male impotence, postmenopausal symptoms in women), dihydrotestosterone (hypogonadism, muscle wasting), dehydroepiandro-sterone (muscle wasting, fat reduction, fitness); estrogens (postmenopausal symptoms, birth control), such as, for example, 17 beta-estradiol, estradiol-3,17-diacetate, estradiol-3-acetate, estradiol-17-acetate, estradiol-3,17-valerate, estradiol-3-valerate, estradiol-17-valerate, ethinyl estradiol, estrone; progesterones (prevent endometriosis, prevent endometrial cancer, control habitual abortion, suppress or synchronize ovulation. promote hair growth), such as, for example, progesterone (preg-4-ene-3,20-dione), norethindrone, norgestrieone, norgestadienone, norgestrel, norgestimate, progestogenic acid, dihydroprogesterol, nomagesterol.

In the above-listed exemplary steroidal reproductive agents, the androgen hormones may be used in any of its known or newly-developed forms, such as, for example, acetate, propionate, 17-beta-cyclopentane-propionate, enanthanate, isobutyrate and undeconate. Similarly, the estradiols may additionally be used in any of its known or newly-developed forms, such as, for example, pivalate, propionate, cypionate, benzoate and other esters. Preferred steroidal reproductive agents, based on the current level of knowledge in the pharmacological arts, are testosterone, progesterone and estradiol, in any of the salt or ester forms. More generally, any steroidal reproductive agent approved by the FDA, or a comparable agency responsible for the regulation of pharmaceutical actives outside the US, such as those listed in, for example, the most current edition of U.S. Pharmacopoeia, may be delivered in the delivery system of the present invention.

In another embodiment, the pharmaceutically-active ingredient is a drug used to reduce or stop hair loss and/or stimulate hair growth, non-limiting examples of which include: 2,3-Dihydro-3-hydroxy-2-imino-6-(1-piperidinyl)-4-pyrimidinamine; 6-(5-Methoxy-1-heptyl)bicyclo(3,3,0)octan-3-one; 4-Amino-1-isobutyl-1H-imidazo(4,5-c)quinoline; 1-Cyano-2-methyl-3-(2-(((5-methyl4-imidazolyl)methyl)thio)ethyl)guanidine; anthralin; 5-α-reductase inhibitors, including (5alpha, 17beta)-(1,1-Dimethylethyl)-3-oxo-4-azaandrost-1-ene-17-carboxamide; and other anti-alopecia agents.

In another embodiment, the pharmaceutically-active ingredient is a drug that is a tranquilizer or sedative, non-limiting examples of which include pharmaceutically-acceptable salts of chlordiazepoxide, benactyzine, benzquinamide, flurazepam, hydroxyzine, loxapine and promazine.

In another embodiment, the pharmaceutically-active ingredient is a muscle-relaxant drug, non-limiting examples of which include pharmaceutically-acceptable salts of cinnamedrine, cyclobenzaprine, flavoxate, orphenadrine, papaverine and mebeverine.

Sunscreen actives may be included in the delivery system of the present invention. Approval by a regulatory agency is generally required for inclusion of a sunscreen active in formulations intended for contact with human skin. Accordingly, sunscreen active agents suitable for incorporation in the present invention include those which are currently approved by the US Food and Drug Administration in the Sunscreen Drug Products for Over-The-Counter Human Use Final Monograph as published in the Federal Register on May 21, 1999 at Volume 64, Number 98, pages 27666-27693. Other sunscreen active ingredients are accepted for use in countries outside the US and are also considered to be within the scope of the present invention.

Other pharmaceutically-active ingredients that can be delivered through the delivery system of the present invention are disclosed in U.S. Pat. No. 6,277,892, in Kerdel, et al., Dermatologic Therapeutics (2005), and in Hardman et al., Goodman & Gilman's: The Pharmacological Basis of Therapeutics (10^{th} Edition, 2001).

Optionally, the delivery system of the present invention may include on or more trace minerals, non-limiting examples of which include: boron, chromium, copper, fluoride, iodine, lithium, magnesium, manganese, molybdenum, selenium, silicon, vanadium, and zinc.

Increased Fragrance Diffusivity and Longevity

The delivery system of the present invention increases the fragrance diffusivity and longevity such that the desired fragrance is organoleptically perceptible for an extended period of time. Fragrance longevity can be measured by analytical methods known to those skilled in the art including solid phase microextraction (SPME), gas chromatography—mass spectroscopy and are described in R. Marsil (ed.) Flavor, Fragrance, and Odor Analysis (2002). Diffusivity can be measured through weight loss due to evaporation of fragrance components versus time, and more subjective methods using trained odor sensing experts including by the methods described in U.S. Patent Publication 2002/0086804 and U.S. Pat. No. 6,703,011.

Dermal Penetration

The delivery system of the present invention increases dermal penetration and, concomitantly, the duration of therapeutic activity. Analysis of enhanced dermal penetration can be accomplished by methods well-known to those skilled in the art, including Franz cell diffusion which quantitatively measures the rate at which agents diffuse or permeate the skin layers. See, e.g., U.S. Patent Publication No. 2001/0031281 and U.S. Pat. No. 4,560,553. The enhanced dermal penetration can also be measured indirectly by the clinician in terms of improvements in the condition being treating.

The following examples are further illustrative of the present invention. The components and specific ingredients are presented as being typical, and various modifications can be derived in view of the foregoing disclosure within the scope of the invention. All percentages, ratios and proportions herein are by weight, unless otherwise specified. All temperatures are in degrees Celsius unless otherwise specified.

### EXAMPLES

AHA Moisturizer

| | | | |
|---|---|---|---|
| A | Water | Deionized Water | 62.0200 |
| | Sclerotium Gum | Amigel (Alban Muller-Tri-K) | 0.5000 |
| | Xanthan Gum | Keltrol (Kelco) | 0.2000 |
| | Methylparaben | Methylparaben | 0.2000 |
| | Disodium EDTA | Dissolvine NA2X (Akzo) | 0.0800 |
| | Glycerin | Glycerine 99.5% | 5.0000 |
| | Butylene Glycol | 1,3-Butylene Glycol (Ashland) | 3.0000 |
| | Panthenol | Liquid DL-Panthenol 50% (DSM) | 1.0000 |
| B | Cyclopentasiloxane | Dow Corning 245 (Dow Corning) | 10.0000 |
| | Cyclopentasiloxane | SF 1214 (G.E. Silicones) | 5.0000 |
| | (and) Dimethicone | | |
| | Dimethicone | Dow Corning 200, 350 cs. | 1.0000 |
| | Propylparaben | Propylparaben | 0.1000 |
| | Phenoxyethanol | Emeressence 1160 (Cogins) | 2.7000 |
| | Salicylic Acid | Salicylic Acid powder, USP/NF | 0.5000 |
| | Glyceryl Stearate (and) | Simulsol 165 (Seppic) | 1.5000 |
| | PEG-100 Stearate | | |
| | Cetearyl Alcohol (and) | Polawax (Croda) | 2.0000 |
| | Polysorbate 60 | | |
| C | Glycolic Acid | Glypure Glycolic Acid, 70% | 4.0000 |
| D | Sodium Hydroxide | Sodium Hydroxide pellets, | 0.4000 |
| | | USP/NF | |
| E | Hydrogen Peroxide | Hydrogen Peroxide, 35% | 0.3000 |
| F | Essential Oil | Essential Oil Blend | 0.5000 |
| | | #6500185 (Bell) | |

Meter deionized water into the processing tank. Sprinkle in Amigel. Mix until completely dispersed. Sprinkle in Keltrol. Heat to 80° C. Mix until uniform. Add to the main tank. Mix until uniform. Cool to 40° C. Add Part C. Mix until uniform. Premix Part D with an equal amount of deoinized water. Add to the main tank. Mix until uniform. Cool to 25° C. Add Part E. Mix until uniform. Add Part F. Mix until uniform.

Clotrimazole Cream

| | | | |
|---|---|---|---|
| A | Water (Aqua) | Deionized Water | 54.9500 |
| | Magnesium Aluminum | Veegum HV (R. T. Vanderbilt) | 1.0000 |
| | Silicate | | |
| | Xanthan Gum | Keltrol CG-T (C. P. Kelco) | 0.3000 |
| | Methylparaben | Methylparaben | 0.2000 |
| B | Propylparaben | Propylparaben | 0.0500 |
| | Dicaprylyl Maleate | Bernel Ester DCM (Bernel/Alzo) | 4.5000 |
| | Simmondsia chinesis | Isojojoba 35 (Desert Whale) | 2.0000 |
| | (jojoba) butter | | |
| | Helianthus annuus | Florasun 90 (Floratech) | 1.0000 |
| | (sunflower) Seed Oil | | |
| | Isohexadecane | Permethyl 101A (Presperse) | 4.5000 |
| | Cetearyl Alcohol | Lanette O (Cognis) | 2.5000 |
| | Glyceryl Stearate (and) | Simulsol 165 (Seppic) | 2.5000 |
| | PEG-100 Stearate | | |
| | Phenoxyethanol | Emeressence 1160 (Cognis) | 2.7000 |
| C | PEG-4 | Carbowax PEG-200 | 7.0000 |
| | | (Dow Chem.) | |
| | Triclosan | Irgasan DP-300 (Ciba) | 0.1000 |
| | Clotrimazole | Clotrimazole | 1.0000 |
| | Salicylic Acid | Salicylic Acid, powder, | 0.5000 |
| | | USP/NF | |
| D | Urea | Urea | 10.0000 |
| E | Glycolic Acid | Glypure 70% Glycolic Acid | 4.0000 |
| | Sodium Hydroxide | Sodium Hydroxide, pellets, | 0.1000 |
| | | USP/NF | |
| F | Papain | Papain | 0.1000 |
| | Dipotassium | OriStar DPG (Orient Stars) | 0.1000 |
| | Glycyrrhizate | | |
| | Tocopheryl Acetate | Vitamin E Acetate (BASF) | 0.1000 |
| | Viis Vinifera (Grape) | Acitiphyte of Grape Seed BG50 | 0.1000 |
| | Seed | | |
| | Extract (and) Water | (Active Organics) | 0.1000 |
| | (Aqua) (and) Butylene | | |
| | Glycol | | |
| | Sodium PCA | Ajidew N-50 | 0.1000 |
| | Proline | Proline | 0.1000 |
| | Essential Oil Blend | Essential Oil Blend | 0.5000 |
| | | #6500185 (Bell) | |

Meter deionized water into the processing tank, reserving 15% for later addition. Sprinkle in Veegum HV. Mix for 20 minutes until uniform. Sprinkle in Keltrol CG-T. Mix until completely dispersed. Heat to 80° C. Add Methylparaben. At 80° C., add Part B ingredients in the order given, mixing well after each addition. Cool to 50° C. Add Part C ingredients. Mix until uniform. Cool to 40° C. In a separate tank, mix part D with the remaining 15% of water. Mix until uniform. Add to the main tank Add Part E ingredients in the given order. Mix until uniform. Cool to 35° C. Add Part F ingredients. Mix until uniform.

Moisturizer SPF 15

| | | | |
|---|---|---|---|
| A | Water Aqua | Deionized Water | 66.3500 |
| | Magnesium Aluminum | Veegum Ultra (R. T. Vanderbilt) | 0.8000 |
| | Silicate | | |
| | Xanthan Gum | Keltrol (C. P. Kelco) | 0.3000 |
| | Panthenol | Liquid DL-Panthenol 50% | 0.2000 |
| | | (DSM) | |
| | Butylene Glycol | 1,3-Butylene Glycol (Ashland) | 3.0000 |
| | Methylparaben | Methylparaben | 0.2000 |
| | Propylparaben | Propylparaben | 0.0500 |
| B | Dimethicone | Dow Corning 200, 350cs | 1.0000 |
| | | (Dow Corning) | |
| | Cetyl phosphate | Amphisol A (DSM Nutritional) | 1.0000 |
| | Glyceryl Stearate (and) | Simulsol 165 (Seppic) | 3.0000 |
| | PEG-100 Stearate | | |
| | Cetyl Alcohol | Lanette 16 (Cognis) | 3.0000 |
| | Neopentyl Glycol | Minno 21 (Bernel/Alzo Intl) | 4.0000 |
| | Diethylhexanoate | | |
| | (and) Neopentyl Glycol | | |
| | Diisostearate | | |
| | Phenoxyethanol | Emmeressence 1160 (Cognis) | 2.7000 |
| | Ethylhexyl | Parsol MCX (DSM Nutritional) | 7.5000 |
| | Methoxycinnamate | | |
| | (Octinoxate) | | |
| | Zinc Oxide (and) | Z-Cote HP-1 | 2.0000 |
| | Triethoxycaprylylsilane | | |
| | C₁₂₋₁₅ Alkyl Benzoate | Finsolv TN (Finetex) | 2.0000 |
| | Salicylic Acid | Salicylic Acid, powder, USP/NF | 0.5000 |
| | Linoleic Acid | Emersol 315 (Cognis) | 0.1000 |
| C | Sodium Hydroxide | Sodium Hydroxide, pellets, | 0.1900 |
| | | USP/NF | |
| D | Glycine Soja (Soybean) | Flavosterone SB (Ichimaru) | 1.0000 |
| | Protein (and) Water (and) | | |
| | Butylene Glycol | | |
| | Punica Granatum | Pomegranate 10% extract | 0.1000 |
| | Extract (and) | in Butylene Glycolo (Premier) | |
| | Butylene Glycol | | |
| | Hydrogen Peroxide | Hydrogen Peroxide, | 0.0100 |
| | | 35% Solution | |
| | Lactic Acid | Lactic acid, Hi-Pure 90 (Purac) | 0.5000 |
| | Essential Oil Blend | Essential Oil Blend | 0.5000 |
| | | #6500185 (Bell) | |

Meter deionized water into the processing tank. Sprinkle in Veegum Ultra. Mix for 20 minutes. Sprinkle in Keltrol. Mix until uniform. Heat to 80° C. Mix until uniform. Homogenize. Add to the main tank. Mix for 20 minutes until uniform. Cool to 40° C. Add Part D ingredients. Mix until uniform.

Blemish Control Moisturizer

| | | | |
|---|---|---|---|
| A | Water (Aqua) | Deionized Water | 66.6000 |
| | Selerotium Gum | Amigel (Alban-Muller-Tri-K) | 0.5000 |
| | Xanthan Gum | Keltrol (Kelco) | 0.2000 |
| | Methylparaben | Methylparaben | 0.2000 |
| | Disodium EDTA | Dissolvine | 0.0500 |
| | Glycerin | Glycerine 99.5% | 5.0000 |
| | Butylene Glycol | 1,3-Butylene Glycol (Ashland) | 3.0000 |
| | Panthenol | Liquid DL-Panthenol 50% (DSM) | 1.0000 |
| B | Cyclopentasiloxane | Dow Corning 245 (Dow Corning) | 10.0000 |
| | Cyclopentasiloxane | SF 1214 (GE Silicones) | 5.0000 |
| | (and) Dimethicone | | |
| | Dimethicone | Dow Corning 200, 350 cs. | 1.0000 |
| | | (Dow Corning) | |
| | Propylparaben | Propylparaben | 0.1000 |
| | Phenoxyethanol | Emeressence 1160 (Cognis) | 2.7000 |
| | Glyceryl Stearate (and) | Simulsol 165 (Seppic) | 1.5000 |
| | PEG-100 Stearate | | |
| | Cetearyl Alcohol (and) | Polawax (Croda) | 2.0000 |
| | Polysorbate 60 | | |
| C | Salicylic Acid | Salicylic Acid, powder, USP/NF | 0.5000 |
| D | Sodium Hydroxide | Sodium Hydroxide, pellets | 0.1500 |
| | | USP/NF | |
| E | Essential Oil Blend | Essential Oil Blend | 0.5000 |
| | | #6500185 (Bell) | |

Meter deionized water into the processing tank. Sprinkle in Amigel. Mix until completely dispersed. Sprinkle in Keltrol. Heat to 80° C. Add the remaining Part A ingredients. Mix until uniform. In a separate tank, heat Part B ingredients to 80° C. Mix until uniform. Add to the main tank. Mix until uniform. Cool to 50° C. Add Part C. Mix until uniform. Premix Part D with an equal amount of deionized water. Add to the main tank. Mix until uniform. Cool to 40° C. Add Part E. Mix until uniform.

Extended Fragrance Delivery Vehicle

| | | | |
|---|---|---|---|
| A | Water (Aqua) | Deionized Water | 69.1400 |
| | Acrylates/C₁₀₋₃₀ | Pemulen TR-1(Noveon) | 0.1800 |
| | Alkyl Acrylate | | |
| | Crosspolymer | | |
| | Tetrasodium EDTA | Dissolvine 220 (Akzo) | 0.0500 |
| | Aminomethyl Propanol | AMP-95 (Dow Chem.) | 0.9000 |
| B | Phenoxyethenol | Emeressence 1160 (Cognis) | 2.7000 |
| | Salicylic Acid | Salicylic Acid, powder, USP | 0.5000 |
| C | Propylene Glycol | Hetester PHA (Bernel/Alzo) | 2.0000 |
| | Isoceteth-3 Acetate | | |
| | Methylheptyl Isostearate | Beantree (Bernel/Alzo) | 2.0000 |
| | Cetyl Ethylhexanoate | Bernel Ester CO | 1.0000 |
| | | (Bernel/Alzo) | |
| | Glyceryl Stearate (and) | Simulsol 165 (Seppic) | 0.0100 |
| | PEG-100 Stearate | | |
| | Mangifera indica (Mango) | Mango Butter (Premier) | 0.0100 |
| | seed butter | | |
| | Olea europaea (Olive) oil | Olive Butter (Premier) | |
| | Tocopheryl Acetate | Vitamin E Acetate (BASF) | |
| D | Alcohol Denat. | SD Alcohol 40-B | 20.0000 |
| E | Essential Oil Blend | Essential Oil Blend | 0.5000 |
| | | #6500185 (Bell) | |

Meter deionized water into the processing tank. Sprinkle in Pemulen TR-1. Mix until completely dispersed. Heat to 75° C. Add the remaining Part A ingredients. Mix until uniform. Add premixed Part B. Mix until uniform. In a separate tank, heat Part C ingredients to 75° C. Mix until uniform. Add to the main tank. Mix until uniform. Cool to 25° C. Add Part D. Mix until uniform. Add Part E. Mix until uniform.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. An oil-in-water emulsion topical delivery system comprising (i) an oil phase; (ii) an aqueous phase; (iii) phenoxyethanol at a concentration of from about 2.0% to about 2.7% based on the total weight of the composition; (iv) an effective exfoliating amount of a hydrophobic hydroxycarboxylic acid selected from the group consisting of orthohydroxybenzoic acid, hydroxycarboxylic acids containing a C₁₂-C₂₄ fatty acid esterified to the alpha carbon hydroxyl group, hydroxycarboxylic acids containing a C₁₂-C₂₄ fatty alcohol esterified to a carboxyl group; (v) a non-ionic emulsifier having an HLB of from about 7 to about 10; (vi) a fragrance composition: and (vii) at least one skin-supporting or dermatopharmaceutically active agent.

2. The topical delivery system of claim 1 where the phenoxyethanol is present at a concentration of from about 2.3% to about 2.7%.

3. The topical delivery system of claim 1 further comprising hydrogen peroxide.

4. The topical delivery system of claim 3 where the hydrogen peroxide is present at a concentration of less than about 3% based on the total weight of the composition.

5. The topical delivery system of claim 4 further comprising a hydrogen peroxide stabilizer selected from the group consisting of amphoteric surfactants, dimethyl amine oxides, chelating agents, tricarboxylic α-hydroxy acids.

6. The topical delivery system of claim 5 where the chelating agent is selected from the group consisting of mono- di-, tri- and tetra-acetic acid derivatives of ethylene diamine.

7. The topical delivery system of claim 6 where the chelating agent is a tetra-acetic acid derivative of ethylene diamine.

8. The topical delivery system of claim 6 where the chelating agent is present at concentration of from about 0.05% to about 0.1% based on the total weight of the composition.

9. The topical delivery system of claim 5 where the tricarboxylic hydroxyacid is 2-hydroxy-1,2,3-propanetricarboxylic acid.

10. The topical delivery system of claim 1 wherein the pH of the topical delivery system is from bout 1.5 to about 2.5 pH units lower than the average pH of the acid mantle of the skin.

11. (canceled)

12. The topical delivery system of claim 1 further comprising a hydrophilic hydroxycarboxylic acid.

13. The topical delivery system of claim 12 where the hydrophilic hydroxycarboxylic acid has a hydroxyl group covalently bonded to the alpha carbon of a carboxylic acid.

14. The topical delivery system of claim 12 where the hydrophilic hydroxycarboxylic acid is selected from the group consisting of 2-hydroxyethanoic acid; 2-hydroxypropanoic acid; 2-hydroxy-2-phenylethanoic acid; 2-hydroxy-1,4-butanedioc acid; 2,3-dihydroxy-1,4-butanedioc acid; 2-hydoxy-,1,2,3-propanetricarboxylic acid.

15. The topical delivery system of claim 12 where the hydrophilic hydroxycarboxylic acid conforms to the formula HOCH₂[CH(OH)]ₙC(═O)OH, where n is an integer from 1 to 10.

16. The topical delivery system of claim 12 wherein the pH of the topical delivery system is from bout 1.5 to about 2.5 pH units lower than the average pH of the acid mantle of the skin.

17-21. (canceled)

22. The topical delivery system of claim 1 where the hydrophobic hydroxycarboxylic acid is orthohydroxybenzoic acid.

23. The topical delivery system of claim 22 where the hydrophobic hydroxycarboxylic acid is present at a concentration of at least about 0.5%.

24-27. (canceled)

28. The topical delivery system of claim 1 wherein the skin-supporting or dermatopharmaceutically active ingredient is selected from the group consisting of agents that reduce the appearance of signs of aging, including fine lines and wrinkles, age spots; amino acids; essential fatty acids; glycosaminoglycans; inhibitors of enzymes that breakdown collagen or elastin; stimulators of collagen or elastin synthesis; antioxidant agents; anti-inflammatory agents; anti-erythemal agents; anti-acne agents; sebum modulators; exfoliating agents; anti-seborrheic agents; antimicrobial agents; anthelmintic agents; skin bleaching and skin lightening agents; anti-cellulite agents: agents that block or absorb ultraviolet radiation and protect the skin from photodamage; agents that promote hair growth; agents that stop or reduce hair loss; hair removal agents; anti-dandruff agents; anesthetic agents; analgesics; tranquilizers; sedatives; muscle relaxants; vasodilators; vasoconstrictors; nitric oxide releasing substances; immunomodulators; peptides, lipopeptides, and derivatives thereof; hormones; astringents; moisturizers; ceramides; hyaluronan and its derivatives; alpha-lipoid acid; vitamins; minerals; an essential oil; and combinations thereof.

29-101. (canceled)

102. The topical delivery system of claim 1 further comprising a non-ionic co-emulsifier having an HLB of from about 8 to about 11.

103-104. (canceled)

105. The topical delivery system of claim 1 wherein the fragrance component is an undiluted essential oil.

106-107. (canceled)

108. The topical delivery system of claim 105 wherein the undiluted essential oil is present at a concentration of at least about 0.1%.

109-110. (canceled)

111. A method for treating a pathophysiologic condition selected from the group consisting of dermatologic conditions, inflammatory conditions, immuno-suppressed conditions, infectious conditions, and disambiguation comprising administering a therapeutically-effective amount of the composition of claim 1 to a person in need thereof.

112-119. (canceled)
